(19) United States
(12) Patent Application Publication
(10) Pub. No.: US 2007/0243101 A1
(43) Pub. Date: Oct. 18, 2007

(54) Cleaning composition with agricultural crop solvent and hydrogen peroxide

(76) Inventor: Louis B. Johnson, Troy, AL (US)

Correspondence Address: CLARK & BRODY, 1090 VERMONT AVENUE, NW, SUITE 250, WASHINGTON, DC, 20005, US

(21) Appl. No.: 11/808,224

(22) Filed: Jun. 7, 2007

Related U.S. Application Data
(63) Continuation of application No. 10/934,361, filed on Sep. 7, 2004, now abandoned.

(51) Int. Cl. A61L 9/00

(52) U.S. Cl. 422/5; 510/372

(57) ABSTRACT

A cleaning composition and method of use uses a solvent derived from an agricultural crop, one or more non-ionic surfactants, one or more anionic surfactants, an anti-oxidant, an optional hydrotrope, and hydrogen peroxide, with the balance being deionized or distilled water. The agricultural crop is preferably soybeans or corn and the solvent is preferably an ester derived from the crop.

This application is a continuation of application Ser. No. 10/934,361, filed on Sep. 7, 2004, which is herein incorporated by reference in its entirety.

### FIELD OF THE INVENTION

The present invention is directed to a cleaning composition, and in particular, to a composition including a solvent derived from an agricultural crop such as soy or corn, hydrogen peroxide, and surfactants for hydrogen peroxide stabilization.

### BACKGROUND ART

In the prior art, hydrogen peroxide is a desirable component of cleaning preparations. However, it is also an unstable compound, and its use in a cleaning composition requires fine tuning in order that the composition remains stable over time, and that the hydrogen peroxide does not break down.

One such composition is disclosed in U.S. Pat. No. 6,316,399 to Melikyan et al. This patent describes a composition combining a terpene such as D-limonene and hydrogen peroxide and a number of surfactants. The aim of this patent is to provide a composition that has high stability over long periods of time. This aim is accomplished by using a terpene, an anti-oxidant, two anionic surfactants, a non-ionic surfactant, hydrogen peroxide, and deionized water. One of the anionic surfactants acts as an emulsifier and a cleaning surfactant, whereas the other anionic surfactant acts as a wetting agent, surface tension reducer, and hydrotrope.

One problem with these types of cleaning compositions is the use of a terpene such as D-limonene as a solvent. Terpenes are disadvantageous in that they have a strong odor, are high in volatile organic compounds (VOC's), can be an irritant to the skin, and have a high flashpoint. In light of these deficiencies, a need exists to provide a cleaning composition that is able to use the benefits of hydrogen peroxide but without the drawbacks of terpenes.

The present invention responds to this need by the discovery of a cleaning composition that uses a solvent that is derived from agricultural crops, e.g., vegetable oil esters, ethyl lactate as derived from corn or mixtures thereof.

### SUMMARY OF THE INVENTION

It is a first object of the present invention to provide an improved cleaning composition that uses a solvent derived from agricultural crops.

Another object of the invention is a cleaning composition that has effective combinations of anionic and non-ionic surfactants and a hydrotrope to ensure solubilization, emulsification, and suspension of components of the composition.

Other objects and advantages of the present invention will become apparent as a description thereof proceeds.

In satisfaction of the foregoing objects and advantages, the present invention provides an improved cleaning composition consisting essentially of a solvent derived from one or more agricultural crops in an amount ranging between 0.01-30.0% by weight; an anti-oxidant in a finite amount between zero and 4.0% by weight; a water soluble co-solvent between zero and up to 10.0% by weight; one or more anionic surfactants, either an alkali metal salt of a linear alkylbenzene sulfonic acid in an amount corresponding to 1.2 parts per 1.0 part of the vegetable crop solvent component, or an alkali metal salt of an alkyl sulfonate in an amount of 0.8 parts per 1.0 part of the vegetable crop component; one or more non-ionic surfactants, each in an amount between 0.5 and 7.0% by weight; a hydrotrope from zero to an effective amount for enhanced solubilization/suspension; an amount of a thickener from zero and up to 5.0% by weight; hydrogen peroxide in an amount between 2.0-75.0% by weight, wherein the hydrogen peroxide amount can be based on a solution of 35% concentration or other concentration if so desired; and the balance deionized water.

The solvent can be any type of a solvent that is derived from one or more agricultural crops and is considered to be “green” or environmentally friendly. Examples include a methyl ester of soy, ethyl lactate, which is an ester of lactic acid, mixtures thereof, esters of other crops such as rapeseed, linseed, sunflower, canola, and the like.

The anionic surfactant can be either or both of the alkali metal salt of a linear alkylbenzene sulfonic acid and the alkali metal salt of an alkyl sulfonate. When using the alkali metal salt of a linear alkylbenzene sulfonic acid, it is preferably an isopropylamine salt of a linear alkylbenzene sulfonic acid. When using the alkali metal salt of an alkyl sulfonate, it is preferably sodium 1-octane sulfonate.

When using the optional co-solvent, a glycol ether such as ethylene glycol monobutyl ether can be used in effective amounts.

When employing the optional hydrotrope in an effective amount to increase the solubilization of the various components of the formulation, a preferred hydrotrope is sodium xylenesulfonate.

The one or more non-ionic surfactants are preferably alcohol ethoxylates. In a more preferred embodiment, two non-ionic surfactants are used, one having a higher degree of ethoxylation, e.g., at least 9.0 or greater, with a target of around 9.5 and one having a lower degree of ethoxylation, around 6.0 or less, with a target of around 6.0.

The invention also entails a method of using the cleaning composition to eliminate odors, to disinfect, to clean and bleach, to remove mildew, and/or remove stains.

### DESCRIPTION OF THE PREFERRED EMBODIMENTS

The present invention is an improvement in cleaning compositions using a terpene and hydrogen peroxide. The inventor has discovered that effective cleaning composition can be made by the use of a solvent derived from agricultural crops in replacement for the terpene solvent, and especially d-limonene derived from oranges as used in the prior art cleaning compositions. The use of these types of “green” solvents offers a significant number of advantages over cleaning compositions using terpene. For example, the types of solvents contemplated as part of the invention are basically odorless and they do not typically produce the VOCs that emanate from terpenes. The flash point of these solvents is higher than terpene. For example, for one preferred solvent, a methyl ester of soy solvent has a flash point around 300° C. as compared to the 120° C. flash point of d-limonene. These “green” solvents are also mild to the skin, and as opposed to d-limonene, which can be irritating to the skin.

Besides the use of an effective amount of the solvent derived from agricultural crops, the cleaning composition can use one or more anionic surfactants in combination with one or more non-ionic surfactants, an anti-oxidant, hydrogen peroxide, and deionized water. In certain formulations, due to a high amount of hydrogen peroxide, the deionized water component may be zero. The formulation can also employ an additional solvent, a thickener, and a hydrotrope to enhance suspension and solubilizing of the various components.

The following table outlines the components of the composition, in terms of weight percent of the compositional solution.

TABLE 1

| | range in weight percent |
|---|---|
| | of component based on |
| Components | entire solution weight |
| an agricultural crop | 0.01-30.0% |
| derived solvent¹ | |
| an anti-oxidant² | a finite amount between |
| | zero and 4.0% |
| a water soluble co- | zero and up to 10.0% |
| solvent³ | |
| one or more anionic | 1.2 parts of the A-type |
| surfactants, either | to 1.0 part of the |
| an A type or B type | solvent component |
| anionic surfactant⁴ | and/or 0.8 parts of the |
| | B-type to 1.0 part of |
| | the solvent component |
| one or more non- | 0.5-7.0% of each |
| ionic surfactants⁵ | surfactant |
| hydrogen peroxide | 2.0-75.0% |
| (35%) | |
| a thickener⁶ | zero and if present, |
| | 0.5 to 5.0% |
| a hydrotrope⁷ | zero and, if present, |
| | 0.5-5.0% |
| deionized water | balance to make 100% |

¹The solvent as generically noted above is preferably a soy-based solvent such as a methyl ester of soy or a soy-containing solvent such as the Vertec Bio Gold that contains ethyl lactates from corn and esters of soy. Solvents such as ethyl lactate alone can also be employed, this type of solvent known commercially as Vertec Bio EL.
²The anti-oxidant is a food grade anti-oxidant such as butylated hydroxy anisole blend with other materials.
³The water soluble co-solvent can be a glycol ether such as ethylene glycol monobutyl ether.
⁴The A type surfactant is an alkali metal salt of a linear alkylbenzene sulfonic acid. The B type surfactant is an alkali metal salt of an alkyl sulfonate.
⁵The non-ionic surfactants are alcohol ethoxylate. When using two non-ionic surfactants one has at least 9 moles of ethoxylation, e.g., Neodol 9.5, with the other having around 6 moles or less of ethoxylation.
⁶The thickener can be a polyacrylate-polyalcohol polymer or an equivalent thereto, with one commercial type being THIX.
⁷The hydrotrope is optional, but when used, a sodium xylenesulfonate is preferred, commercially known as SXS.

Examples of the solvent for the inventive cleaning composition include the soy gold solvents (methyl ester of soy) from soygold.com, Vertec Bio Gold from Vertecbiosolvents.com (a blend of a corn derived solvent as ethyl lactate and methyl ester of soy), and ethyl lactate. While soy and corn derived solvents are exemplified, it should be understood that other agricultural crops could also be used as a source for the solvent, e.g., seeds such as canola, sunflower, rapeseed, so as to produce canola oil esters, sunflower oil esters, linseed oil esters, rapeseed oil esters, and the like, including ethyl and methyl esters of the particular agricultural crop. An agricultural crop is considered to be the yield from plants or flowers grown in mass in field conditions wherein the plant or flower is harvested on a seasonal basis to obtain at least the plant components that produce an oil as a source for the solvent for use in the invention. These crops are different from the production from orchards or the like, wherein just the fruit of trees is harvested.

More details of other components are as follows:

One or Both of the Type A or B Anionic Surfactants

As stated above, one or more types of anionic surfactants can be employed in conjunction with the hydrogen peroxide and other components of the invention. One preferred surfactant as Type A is an isopropylamine salt of the linear alkylbenzene sulfonic acid. A preferred Type B anionic surfactant is sodium 1-octane sulfonate.

One commercial formulation of the Type A surfactant listed above is BIOSOFT-411. This type A surfactant is available from a number of suppliers, e.g. Stepan, and is also sold under a different trade name but still identified as a match with BIOSOFT-411. Likewise, one commercial formulations of the Type B surfactant is Bioterge PAS-8S and this is available from one or more suppliers, either under this trade name or under another trade name known to be an equivalent to Bioterge PAS-8S. As stated above, the type A and type B components can be combined

In order to maintain the stability of the hydrogen peroxide, a single anionic surfactant can be employed or first and second anionic surfactants can be used as is suggested in the Melikyan et al. patent.

Non-Ionic Surfactants

The non-ionic surfactant can have its number of carbon atoms vary, with a preferred range being between 10-15 carbon atoms. A preferred HLB value is 13.1. In this regard, one commercial formulation for this type of surfactant is Neodol 25-9, which is available from a number of suppliers, whose identities can be obtained by using the world wide web. Other commercial formulations under different trade names are also available as an equivalent to Neodol 25-9. It is preferred to use two non-ionic surfactants when using the soy ester as the solvent for the formulation. The two surfactants should have different moles of ethoxylation so that the formulation has both rinsing capability and adequate emulsification properties. Rinsing capability is the property wherein the formula can be easily rinsed off a surface after application. It has been discovered that using just one non-ionic surfactant such as the Neodol 25-9 provides adequate rinsing properties but does not provide optimum emulsification of a solvent such as methyl soy ester. Thus, it is preferred to have a lower mole surfactant for better emulsification while still retaining the higher mole surfactant, for rinsing capability. It is believed that surfactants having an ethoxylation mole range of at least 9 are adequate for rinsing with surfactants having an ethoxylation of around 6 moles or less are adequate for emulsification. If using one non-ionic surfactant, a nine or greater mole alcohol ethoxylate should be used. Virtually any commercially available alcohol ethoxylate can be used as the one or more non-ionic surfactants provided that the guidelines given above for moles of ethoxylation are used.

Hydrotrope

In certain instances, it is preferred to use a hydrotrope to increase the solubilization/suspension of the components of the formulation, particularly when using the methyl ester of soy as the solvent. This solvent makes it a little more difficult to solubilize the various components, and an effective amount of a hydrotrope improves the overall solubilization. A preferred hydrotrope is sodium xylenesulfonate (commercially available as SXS), but other hydrotropes as would be known in the art could be employed. Using the hydrotrope in the formulation is especially beneficial in instances where high volumes of the formulation will be stored. In these situations, the formulations remain relatively static and separation of the formulation components can occur. This is not the case in lower volume containers, e.g., ones used in the home or sold at the retail level, since they are moved around frequently, with such movement keeping the formulation components suspended/solubilized. While the amount of the hydrotrope should be sufficient to assist in keeping the components solubilized/suspended, a preferred range includes between about 0.5% and 5.0% by weight of the formulation, with a target of around 2-3% by weight.

While Table 1 above outlines the limits of the invention, specific formulations using a methyl ester of soy are detailed below in TABLE 2.

TABLE 2

| Formulation | wt. % | | | | | | | | | |
|---|---|---|---|---|---|---|---|---|---|---|
| component | A | B | C | E | F | G | H | I | J | K |
| deionized | 55.36 | 58.17 | 93.06 | 93.59 | 0.00 | 0.00 | 49.89 | 93.89 | 94.11 | 92.69 |
| water | | | | | | | | | | |
| anti-oxidant | 0.02 | 0.02 | 0.02 | 0.02 | 0.02 | 0.02 | 0.02 | 0.02 | 0.02 | 0.02 |
| glycol ether | 0.00 | 0.00 | 0.00 | 0.00 | 0.00 | 0.00 | 7.00 | 0.58 | 0.58 | 0.58 |
| methyl ester | 7.08 | 7.08 | 1.32 | 1.32 | 7.08 | 7.08 | 6.58 | 0.55 | 0.55 | 0.55 |
| of soy | | | | | | | | | | |
| Biosoft-411 | 8.55 | 0.00 | 1.60 | 0.00 | 8.55 | 0.00 | 7.95 | 0.66 | 0.00 | 0.66 |
| Bioterge | 0.00 | 5.74 | 0.00 | 1.07 | 00.0 | 5.74 | 0.00 | 0.00 | 0.44 | 0.00 |
| PAS-8S | | | | | | | | | | |
| Neodol 25-9 | 6.41 | 6.44 | 0.00 | 1.07 | 6.41 | 6.41 | 5.96 | 0.50 | 0.50 | 0.50 |
| Alcohol | 2.0 | 2.0 | 2.0 | 2.0 | 2.0 | 2.0 | 2.0 | 2.0 | 2.0 | 2.0 |
| ethoxylate | | | | | | | | | | |
| with 6 moles | | | | | | | | | | |
| of | | | | | | | | | | |
| ethoxylation | | | | | | | | | | |
| Sodium | 3.0 | 3.0 | 3.0 | 3.0 | 3.0 | 3.0 | 3.0 | 3.0 | 3.0 | 3.0 |
| xylene- | | | | | | | | | | |
| Sulfonate | | | | | | | | | | |
| Thickener | 0.00 | 0.00 | 0.00 | 0.00 | 0.00 | 0.00 | 0.00 | 0.00 | 1.00 | 1.00 |
| Hydrogen | 22.6 | 22.6 | 2.80 | 2.80 | 72.2 | 72.2 | 22.6 | 2.80 | 2.80 | 4.00 |
| peroxide (35%) | | | | | | | | | | |

Another preferred formulation, in weight percent, of the invention includes both of the anionic surfactants and both non-ionic surfactants, and the hydrotrope, as 7.08% of the methyl ester of soy, 2% of the 6 mole or lower alcohol ethoxylate non-ionic surfactant, 8.55% of the Biosoft 411, 5.74% of the Bioterge Pas 8S, 6.41% of the 9 or higher mole alcohol ethoxylate non-ionic surfactant, 3% of the hydrotrope as SXS or sodium xylenesulfonate, 44.12% of de-ionized water, and 22.6% hydrogen peroxide. When making the compositions of the invention, it is preferred that the ingredients other than water and the peroxide be added together and mixed well. Then the water is added with further mixing. Finally, the hydrogen peroxide is added last with a final mixing. It should be understood that the specific formulations are but examples of the invention, and the various components used in the formulation can range as effective amounts or the ranges specified above.

It should also be understood that this application incorporates by reference the Melikyan et al. patent discussed above, particularly with regard to the use of two anionic surfactants. Also incorporated by reference in its entirety is the disclosure of U.S. Pat. No. 6,939,839 to Johnson. In this regard, the Melikyan patent discloses a number of different concentration levels for different uses in Table 2 thereof, and any of the uses would be applicable with any of the inventive formulations encompassed by Table 1 or the specifics formulations of Table 2 of the instant application.

As noted in the Table 1 and 2 above, the hydrogen peroxide is preferred in a 35% concentration, but other concentrations could be employed.

The thickener is an optional component of the formulation as is the co-solvent. A preferred co-solvent is a glycol ether, more preferably an ethylene glycol monobutyl ether or an equivalent thereto, since these types are effective in removing organic and petroleum soils as a result of the ether linkage.

The cleaning composition can be used virtually for any cleaning use, either for the consumer or in the industrial area. The uses include those known uses disclosed in Table 2 of the Melikyan patent, and any other known uses where cleaning, degreasing, odor and/or mildew elimination, disinfection, and stain removal are needed.

The cleaning composition can also employ a preservative in an amount effective to preserve the formulation and resist bacterial growth. Preferred ranges include from 0.10 to 1% by weight, more preferred ranges include 0.15% to 0.5% with a target of around 0.2-0.3%. Virtually any known preservative can be employed in the formulation, with examples including those using propyl paraben, methyl paraben and diazolidinyl urea, e.g., Germaben II. Another specific preservative that can be used is Dantoguard® manufactured by Lonza, see www.Lonza.com.

As such an invention has been disclosed in terms of preferred embodiments thereof, which fulfills each and every one of the objects of the invention as set forth above, and provides an improved cleaning composition.

Of course, various changes, modifications and alterations from the teachings of the present invention may be contemplated by those skilled in the art without departing from the intended spirit and scope thereof. It is intended that the present invention only be limited by the terms of the appended claims.

What is claimed is:

1. A cleaning composition consisting essentially of:
a solvent derived from an agricultural crop in an amount ranging between 0.01 and 30.0% by weight;
an anti-oxidant in a finite amount between zero and 4.0% by weight;
a water soluble co-solvent between zero and up to 10.0% by weight;
one or more anionic surfactants, either an alkali metal salt of a linear alkylbenzene sulfonic acid in an amount corresponding to 1.2 parts per 1.0 part of the solvent component, and/or an alkali metal salt of an alkyl sulfonate in an amount of 0.8 parts per 1.0 part of the solvent component;
one or more non-ionic surfactants, each in an amount between 0.5 and 7.0% by weight;
hydrogen peroxide in an amount between 2.0-75.0% by weight;
a thickener from zero and up to 5.0% by weight; and
the balance deionized water.

2. The cleaning composition of claim 1, wherein the anionic surfactant is the alkali metal salt of a linear alkylbenzene sulfonic acid.

3. The cleaning composition of claim 1, wherein the anionic surfactant is the alkali metal salt of an alkyl sulfonate.

4. The cleaning composition of claim 2, wherein the alkali metal salt of a linear alkylbenzene sulfonic acid is an isopropylamine salt of a linear alkylbenzene sulfonic acid.

5. The cleaning composition of claim 3, wherein the alkali metal salt of an alkyl sulfonate is sodium 1-octane sulfonate.

6. The cleaning composition of claim 1, wherein the solvent is a methyl ester of soy, ethyl lactate or a mixture thereof.

7. The cleaning composition of claim 1, further comprising glycol ether as the water soluble co-solvent in a finite and effective amount.

8. The cleaning composition of claim 1, wherein two non-ionic surfactants are used, each being an alcohol ethoxylate, one having at least 9.0 moles of ethoxylation and the other having 6.0 moles or less of ethoxylation.

9. The cleaning composition of claim 1, wherein the anti-oxidant is a food grade anti-oxidant.

10. The cleaning composition of claim 1, further comprising an effective amount of a hydrotrope for enhancing solubilization of the composition.

11. The cleaning composition of claim 10, wherein the hydrotrope is a sodium xylenesulfonate in an amount between 0.5 and 5.0% by weight.

12. The cleaning composition consisting essentially of:
a solvent derived from an agricultural crop in an amount ranging between 0.55 and 7.08% by weight;
an anti-oxidant in a finite amount up to 0.02% by weight;
one or more anionic surfactants, either an isopropylamine salt of a linear alkylbenzene sulfonic acid in an amount corresponding to 1.2 parts per 1.0 part of the solvent component, and/or sodium 1-octane sulfonate in an amount of 0.8 parts per 1.0 part of the solvent component;
an alcohol ethoxylate non-ionic surfactant having at least 9.0 moles of ethoxylation in an amount between 0.5 and 7.0% by weight, and an alcohol ethoxylate non-ionic surfactant having 6.0 or less moles of ethoxylation in an amount between 0.5 and 7.0% by weight;
a hydrotrope in an amount between 0.5 and 5.0% by weight;
hydrogen peroxide in an amount between 2.8-72.2% by weight; and
the balance deionized water.

13. The cleaning composition of claim 12, wherein the anionic surfactant is the isopropylamine salt of a linear alkylbenzene sulfonic acid.

14. The cleaning composition of claim 12, wherein the anionic surfactant is sodium 1-octane sulfonate.

15. The cleaning composition of claim 12, wherein the non-ionic surfactants have 6 and between 9.0 and 9.5 moles of ethoxylation, respectively.

16. In a method of cleaning a surface to eliminate odors, to disinfect, to clean and bleach, to remove mildew, and/or remove stains, the improvement comprising applying the cleaning composition of claim 1 to the surface.

17. The method of claim 16, wherein the surface is a hard surface.

18. In a method of cleaning a surface to eliminate odors, to disinfect, to clean and bleach, to remove mildew, and/or remove stains, the improvement comprising applying the cleaning composition of claim 12 to the surface.

19. The method of claim 18, wherein the surface is a hard surface.
